(19) United States
(12) Patent Application Publication
(10) Pub. No.: US 2018/0008717 A1
(43) Pub. Date: Jan. 11, 2018

(54) PHARMACEUTICAL COMPOSITION WITH ANALGESIC AND ANTI-INFLAMMATORY ACTIVITY

(71) Applicants: Iryna Kravchenko, Santa Clara, CA (US); Mariia Nesterkina, Santa Clara, CA (US); Sergiy Lozovsky, Santa Clara, CA (US)

(72) Inventors: Iryna Kravchenko, Santa Clara, CA (US); Mariia Nesterkina, Santa Clara, CA (US); Sergiy Lozovsky, Santa Clara, CA (US)

(21) Appl. No.: 15/645,827

(22) Filed: Jul. 10, 2017

Related U.S. Application Data
(60) Provisional application No. 62/360,383, filed on Jul. 10, 2016.

(51) Int. Cl. A61K 31/197; A61K 9/00; A61K 31/085; A61K 31/045; C07C 229/46

(52) U.S. Cl. CPC A61K 47/542; A61K 31/197; A61K 31/085; A61K 31/045; A61K 9/0053; A61K 9/0014; C07C 229/46

(57) ABSTRACT

A composition comprising a compound having a formula I:

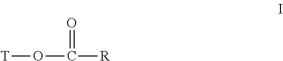

wherein T is derivative of a terpene and R is a derivative of a neurotransmitter acids, wherein the neurotransmitter acids are selected form GABA, glycine, and taurine. Pharmaceutical formulations for oral and topical use as analgesics and anti-inflammatories are also provided. A process for preparing the composition and the formulations are also provided.

### CROSS-REFERENCE TO RELATED APPLICATIONS

The present Utility patent application claims priority benefit of the [U.S. provisional application for patent Ser. No. 62/360,383 entitled “ANALGESIC AND ANTI-INFLAMMATORY ACTIVITY OF TERPENOID ESTERS” filed 10 Jul. 2016 under 35 U.S.C. 119(e). The contents of this related provisional application are incorporated herein by reference for all purposes to the extent that such subject matter is not inconsistent herewith or limiting hereof.

### INCORPORATION BY REFERENCE OF SEQUENCE LISTING PROVIDED AS ATEXT FILE

Not applicable.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection by the author thereof. The copyright owner has no objection to the facsimile reproduction by anyone of the patent document or patent disclosure for the purposes of referencing as patent prior art, as it appears in the Patent and Trademark Office, patent file or records, but otherwise reserves all copyright rights whatsoever.

### BACKGROUND OF THE RELEVANT PRIOR ART

One or more embodiments of the invention generally relate to compositions having analgesic and anti-inflammatory effect. More particularly, certain embodiments of the invention relates to compositions that include esters based on terpenoids and neurotransmitter amino acids having analgesic and anti-inflammatory effect.

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon. Research is ongoing in the pharmaceutical industry for new compositions to be used the treatment of pain and/or inflammation particularly for medications without addictive effect. The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that significant progress has been made in nociception and inflammatory reaction owing to the identification, cloning and characterization of transient receptor potential (TRP) family of cation channels. Typically, TRP channels are divided into seven subfamilies based on their amino acid sequence homology: transient receptor potential canonical (TRPC), TRPV (“V” for vanilloid), TRPM (“M” for melastatin), TRPN, TRPA, TRPP (“P” for polycystic) and TRPML (“ML” for mucolipin), each of which are typically known to be activated by a number of phytochemicals. For example, it is believed that, thymol and carvacrol can activate TRPV1 and TRPV3 ion channels; guaiacol may exert its antinociceptive effects via TRPV1; and menthol was found to be a TRPM8 agonist. Furthermore, it is believed that the abovementioned monocyclic terpenes are active within the central nerrvouce system (CNS) and act as positive allosteric modulators of gamma (γ)-aminobutyric acid (GABA_{A}) receptors showing a variety of effects such as anticonvulsant, analgesic, and nootropic actions. It is believed GABA is well known as the main inhibitory neurotransmitter of the central CNS, and its low levels are responsible for many pain states. Additionally, recent studies have provided strong evidences that GABA_{B} receptors are also localized in the periphery; surprising in this context is the finding of GABA localization at the terminal endings of corneal nociceptors. Thus, the combination of TRP-channel modulators and compounds that bind to GABA receptors is expedient for development of novel drugs possessing a wide range of pharmacological activity.”

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the FIGURES of the accompanying drawings and in which like reference numerals refer to similar elements and in which:

FIG. 1 illustrates an exemplary graph of the anti-inflammatory activity of esters described herein on the model of an allyl isothiocyanate (AITC)-induced edema, in accordance with an embodiment of the present invention.

Unless otherwise indicated illustrations in the figures are not necessarily drawn to scale.

### DETAILED DESCRIPTION OF SOME EMBODIMENTS

The present invention is best understood by reference to the detailed FIGURES and description set forth herein.

Embodiments of the invention are discussed below with reference to the FIGURES. However, those skilled in the art will readily appreciate that the detailed description given herein with respect to these FIGURES is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

All words of approximation as used in the present disclosure and claims should be construed to mean “approximate,” rather than “perfect,” and may accordingly be employed as a meaningful modifier to any other word, specified parameter, quantity, quality, or concept. Words of approximation, include, yet are not limited to terms such as “substantial”, “nearly”, “almost”, “about”, “generally”, “largely”, “essentially”, “closely approximate”, etc.

As will be established in some detail below, it is well settle law, as early as 1939, that words of approximation are not indefinite in the claims even when such limits are not defined or specified in the specification.

For example, see Ex parte Mallory, 52 USPQ 297, 297 (Pat. Off. Bd. App. 1941) where the court said “The examiner has held that most of the claims are inaccurate because apparently the laminar film will not be entirely eliminated. The claims specify that the film is “substantially” eliminated and for the intended purpose, it is believed that the slight portion of the film which may remain is negligible. We are of the view, therefore, that the claims may be regarded as sufficiently accurate.”

Note that claims need only “reasonably apprise those skilled in the art” as to their scope to satisfy the definiteness requirement. See Energy Absorption Sys., Inc. v. Roadway Safety Servs., Inc., Civ. App. 96-1264, slip op. at 10 (Fed. Cir. Jul. 3, 1997) (unpublished) Hybridtech v. Monoclonal Antibodies, Inc., 802 F.2d 1367, 1385, 231 USPQ 81, 94 (Fed. Cir. 1986), cert. denied, 480 U.S. 947 (1987). In addition, the use of modifiers in the claim, like “generally” and “substantial,” does not by itself render the claims indefinite. See Seattle Box Co. v. Industrial Crating & Packing, Inc., 731 F.2d 818, 828-29, 221 USPQ 568, 575-76 (Fed. Cir. 1984).

Moreover, the ordinary and customary meaning of terms like “substantially” includes “reasonably close to: nearly, almost, about”, connoting a term of approximation. See In re Frye, Appeal No. 2009-006013, 94 USPQ2d 1072, 1077, 2010 WL 889747 (B.P.A.I. 2010) Depending on its usage, the word “substantially” can denote either language of approximation or language of magnitude. Deering Precision Instruments, L.L.C. v. Vector Distribution Sys., Inc., 347 F.3d 1314, 1323 (Fed. Cir. 2003) (recognizing the “dual ordinary meaning of th[e] term [“substantially”] as connoting a term of approximation or a term of magnitude”). Here, when referring to the “substantially halfway” limitation, the Specification uses the word “approximately” as a substitute for the word “substantially” (Fact 4). (Fact 4). The ordinary meaning of “substantially halfway” is thus reasonably close to or nearly at the midpoint between the forwardmost point of the upper or outsole and the rearwardmost point of the upper or outsole.

Similarly, the term ‘substantially’ is well recognize in case law to have the dual ordinary meaning of connoting a term of approximation or a term of magnitude. See Dana Corp. v. American Axle & Manufacturing, Inc., Civ. App. 04-1116, 2004 U.S. App. LEXIS 18265, *13-14 (Fed. Cir. Aug. 27, 2004) (unpublished). The term “substantially” is commonly used by claim drafters to indicate approximation. See Cordis Corp. v. Medtronic AVE Inc., 339 F.3d 1352, 1360 (Fed. Cir. 2003) (“The patents do not set out any numerical standard by which to determine whether the thickness of the wall surface is ‘substantially uniform.’ The term ‘substantially,’ as used in this context, denotes approximation. Thus, the walls must be of largely or approximately uniform thickness.”); see also Deering Precision Instruments, LLC v. Vector Distribution Sys., Inc., 347 F.3d 1314, 1322 (Fed. Cir. 2003); Epcon Gas Sys., Inc. v. Bauer Compressors, Inc., 279 F.3d 1022, 1031 (Fed. Cir. 2002). We find that the term “substantially” was used in just such a manner in the claims of the patents-in-suit: “substantially uniform wall thickness” denotes a wall thickness with approximate uniformity.

It should also be noted that such words of approximation as contemplated in the foregoing clearly limits the scope of claims such as saying ‘generally parallel’ such that the adverb ‘generally’ does not broaden the meaning of parallel. Accordingly, it is well settled that such words of approximation as contemplated in the foregoing (e.g., like the phrase ‘generally parallel’) envisions some amount of deviation from perfection (e.g., not exactly parallel), and that such words of approximation as contemplated in the foregoing are descriptive terms commonly used in patent claims to avoid a strict numerical boundary to the specified parameter. To the extent that the plain language of the claims relying on such words of approximation as contemplated in the foregoing are clear and uncontradicted by anything in the written description herein or the FIGURES thereof, it is improper to rely upon the present written description, the FIGURES, or the prosecution history to add limitations to any of the claim of the present invention with respect to such words of approximation as contemplated in the foregoing. That is, under such circumstances, relying on the written description and prosecution history to reject the ordinary and customary meanings of the words themselves is impermissible. See, for example, Liquid Dynamics Corp. v. Vaughan Co., 355 F.3d 1361, 69 USPQ2d 1595, 1600-01 (Fed. Cir. 2004). The plain language of phrase 2 requires a “substantial helical flow.” The term “substantial” is a meaningful modifier implying “approximate,” rather than “perfect.” In Cordis Corp. v. Medtronic AVE, Inc., 339 F.3d 1352, 1361 (Fed. Cir. 2003), the district court imposed a precise numeric constraint on the term “substantially uniform thickness.” We noted that the proper interpretation of this term was “of largely or approximately uniform thickness” unless something in the prosecution history imposed the “clear and unmistakable disclaimer” needed for narrowing beyond this simple-language interpretation. Id. In Anchor Wall Systems v. Rockwood Retaining Walls, Inc., 340 F.3d 1298, 1311 (Fed. Cir. 2003)” Id. at 1311. Similarly, the plain language of claim 1 requires neither a perfectly helical flow nor a flow that returns precisely to the center after one rotation (a limitation that arises only as a logical consequence of requiring a perfectly helical flow).

The reader should appreciate that case law generally recognizes a dual ordinary meaning of such words of approximation, as contemplated in the foregoing, as connoting a term of approximation or a term of magnitude; e.g., see Deering Precision Instruments, L.L.C. v. Vector Distrib. Sys., Inc., 347 F.3d 1314, 68 USPQ2d 1716, 1721 (Fed. Cir. 2003), cert. denied, 124 S. Ct. 1426 (2004) where the court was asked to construe the meaning of the term “substantially” in a patent claim. Also see Epcon, 279 F.3d at 1031 (“The phrase ‘substantially constant’ denotes language of approximation, while the phrase ‘substantially below’ signifies language of magnitude, i.e., not insubstantial.”). Also, see, e.g., Epcon Gas Sys., Inc. v. Bauer Compressors, Inc., 279 F.3d 1022 (Fed. Cir. 2002) (construing the terms “substantially constant” and “substantially below”); Zodiac Pool Care, Inc. v. Hoffinger Indus., Inc., 206 F.3d 1408 (Fed. Cir. 2000) (construing the term “substantially inward”); York Prods., Inc. v. Cent. Tractor Farm & Family Ctr., 99 F.3d 1568 (Fed. Cir. 1996) (construing the term “substantially the entire height thereof”); Tex. Instruments Inc. v. Cypress Semiconductor Corp., 90 F.3d 1558 (Fed. Cir. 1996) (construing the term “substantially in the common plane”). In conducting their analysis, the court instructed to begin with the ordinary meaning of the claim terms to one of ordinary skill in the art. Prima Tek, 318 F.3d at 1148. Reference to dictionaries and our cases indicates that the term “substantially” has numerous ordinary meanings. As the district court stated, “substantially” can mean “significantly” or “considerably.” The term “substantially” can also mean “largely” or “essentially.” Webster's New 20th Century Dictionary 1817 (1983).

Words of approximation, as contemplated in the foregoing, may also be used in phrases establishing approximate ranges or limits, where the end points are inclusive and approximate, not perfect; e.g., see AK Steel Corp. v. Sollac, 344 F.3d 1234, 68 USPQ2d 1280, 1285 (Fed. Cir. 2003) where it where the court said [W]e conclude that the ordinary meaning of the phrase “up to about 10%” includes the “about 10%” endpoint. As pointed out by AK Steel, when an object of the preposition “up to” is nonnumeric, the most natural meaning is to exclude the object (e.g., painting the wall up to the door). On the other hand, as pointed out by Sollac, when the object is a numerical limit, the normal meaning is to include that upper numerical limit (e.g., counting up to ten, seating capacity for up to seven passengers). Because we have here a numerical limit “about 10%”—the ordinary meaning is that that endpoint is included.

In the present specification and claims, a goal of employment of such words of approximation, as contemplated in the foregoing, is to avoid a strict numerical boundary to the modified specified parameter, as sanctioned by Pall Corp. v. Micron Separations, Inc., 66 F.3d 1211, 1217, 36 USPQ2d 1225, 1229 (Fed. Cir. 1995) where it states “It is well established that when the term “substantially” serves reasonably to describe the subject matter so that its scope would be understood by persons in the field of the invention, and to distinguish the claimed subject matter from the prior art, it is not indefinite.” Likewise see Verve LLC v. Crane Cams Inc., 311 F.3d 1116, 65 USPQ2d 1051, 1054 (Fed. Cir. 2002). Expressions such as “substantially” are used in patent documents when warranted by the nature of the invention, in order to accommodate the minor variations that may be appropriate to secure the invention. Such usage may well satisfy the charge to “particularly point out and distinctly claim” the invention, 35 U.S.C. §112, and indeed may be necessary in order to provide the inventor with the benefit of his invention. In Andrew Corp. v. Gabriel Elecs. Inc., 847 F.2d 819, 821-22, 6 USPQ2d 2010, 2013 (Fed. Cir. 1988) the court explained that usages such as “substantially equal” and “closely approximate” may serve to describe the invention with precision appropriate to the technology and without intruding on the prior art. The court again explained in Ecolab Inc. v. Envirochem, Inc., 264 F.3d 1358, 1367, 60 USPQ2d 1173, 1179 (Fed. Cir. 2001) that “like the term ‘about,’ the term ‘substantially’ is a descriptive term commonly used in patent claims to ‘avoid a strict numerical boundary to the specified parameter, see Ecolab Inc. v. Envirochem Inc., 264 F.3d 1358, 60 USPQ2d 1173, 1179 (Fed. Cir. 2001) where the court found that the use of the term “substantially” to modify the term “uniform” does not render this phrase so unclear such that there is no means by which to ascertain the claim scope.

Similarly, other courts have noted that like the term “about,” the term “substantially” is a descriptive term commonly used in patent claims to “avoid a strict numerical boundary to the specified parameter.”; e.g., see Pall Corp. v. Micron Seps., 66 F.3d 1211, 1217, 36 USPQ2d 1225, 1229 (Fed. Cir. 1995); see, e.g., Andrew Corp. v. Gabriel Elecs. Inc., 847 F.2d 819, 821-22, 6 USPQ2d 2010, 2013 (Fed. Cir. 1988) (noting that terms such as “approach each other,” “close to,” “substantially equal,” and “closely approximate” are ubiquitously used in patent claims and that such usages, when serving reasonably to describe the claimed subject matter to those of skill in the field of the invention, and to distinguish the claimed subject matter from the prior art, have been accepted in patent examination and upheld by the courts). In this case, “substantially” avoids the strict 100% nonuniformity boundary.

Indeed, the foregoing sanctioning of such words of approximation, as contemplated in the foregoing, has been established as early as 1939, see Ex parte Mallory, 52 USPQ 297, 297 (Pat. Off. Bd. App. 1941) where, for example, the court said “the claims specify that the film is “substantially” eliminated and for the intended purpose, it is believed that the slight portion of the film which may remain is negligible. We are of the view, therefore, that the claims may be regarded as sufficiently accurate.” Similarly, In re Hutchison, 104 F.2d 829, 42 USPQ 90, 93 (C.C.P.A. 1939) the court said “It is realized that “substantial distance” is a relative and somewhat indefinite term, or phrase, but terms and phrases of this character are not uncommon in patents in cases where, according to the art involved, the meaning can be determined with reasonable clearness.”

Hence, for at least the forgoing reason, Applicants submit that it is improper for any examiner to hold as indefinite any claims of the present patent that employ any words of approximation.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will be described in detail below with reference to embodiments thereof as illustrated in the accompanying drawings.

References to a “device,” an “apparatus,” a “system,” etc., in the preamble of a claim should be construed broadly to mean “any structure meeting the claim terms” exempt for any specific structure(s)/type(s) that has/(have) been explicitly disavowed or excluded or admitted/implied as prior art in the present specification or incapable of enabling an object/aspect/goal of the invention. Furthermore, where the present specification discloses an object, aspect, function, goal, result, or advantage of the invention that a specific prior art structure and/or method step is similarly capable of performing yet in a very different way, the present invention disclosure is intended to and shall also implicitly include and cover additional corresponding alternative embodiments that are otherwise identical to that explicitly disclosed except that they exclude such prior art structure(s)/step(s), and shall accordingly be deemed as providing sufficient disclosure to support a corresponding negative limitation in a claim claiming such alternative embodiment(s), which exclude such very different prior art structure(s)/step(s) way(s).

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” “some embodiments,” “embodiments of the invention,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least some embodiments of the invention” includes the stated particular feature, structure, or characteristic.

References to “user”, or any similar term, as used herein, may mean a human or non-human user thereof. Moreover, “user”, or any similar term, as used herein, unless expressly stipulated otherwise, is contemplated to mean users at any stage of the usage process, to include, without limitation, direct user(s), intermediate user(s), indirect user(s), and end user(s). The meaning of “user”, or any similar term, as used herein, should not be otherwise inferred or induced by any pattern(s) of description, embodiments, examples, or referenced prior-art that may (or may not) be provided in the present patent.

References to “end user”, or any similar term, as used herein, is generally intended to mean late stage user(s) as opposed to early stage user(s). Hence, it is contemplated that there may be a multiplicity of different types of “end user” near the end stage of the usage process. Where applicable, especially with respect to distribution channels of embodiments of the invention comprising consumed retail products/services thereof (as opposed to sellers/vendors or Original Equipment Manufacturers), examples of an “end user” may include, without limitation, a “consumer”, “buyer”, “customer”, “purchaser”, “shopper”, “enjoyer”, “viewer”, or individual person or non-human thing benefiting in any way, directly or indirectly, from use of. or interaction, with some aspect of the present invention.

In some situations, some embodiments of the present invention may provide beneficial usage to more than one stage or type of usage in the foregoing usage process. In such cases where multiple embodiments targeting various stages of the usage process are described, references to “end user”, or any similar term, as used therein, are generally intended to not include the user that is the furthest removed, in the foregoing usage process, from the final user therein of an embodiment of the present invention.

Where applicable, especially with respect to retail distribution channels of embodiments of the invention, intermediate user(s) may include, without limitation, any individual person or non-human thing benefiting in any way, directly or indirectly, from use of, or interaction with, some aspect of the present invention with respect to selling, vending, Original Equipment Manufacturing, marketing, merchandising, distributing, service providing, and the like thereof.

References to “person”, “individual”, “human”, “a party”, “animal”, “creature”, or any similar term, as used herein, even if the context or particular embodiment implies living user, maker, or participant, it should be understood that such characterizations are sole by way of example, and not limitation, in that it is contemplated that any such usage, making, or participation by a living entity in connection with making, using, and/or participating, in any way, with embodiments of the present invention may be substituted by such similar performed by a suitably configured non-living entity, to include, without limitation, automated machines, robots, humanoids, computational systems, information processing systems, artificially intelligent systems, and the like. It is further contemplated that those skilled in the art will readily recognize the practical situations where such living makers, users, and/or participants with embodiments of the present invention may be in whole, or in part, replaced with such non-living makers, users, and/or participants with embodiments of the present invention. Likewise, when those skilled in the art identify such practical situations where such living makers, users, and/or participants with embodiments of the present invention may be in whole, or in part, replaced with such non-living makers, it will be readily apparent in light of the teachings of the present invention how to adapt the described embodiments to be suitable for such non-living makers, users, and/or participants with embodiments of the present invention. Thus, the invention is thus to also cover all such modifications, equivalents, and alternatives falling within the spirit and scope of such adaptations and modifications, at least in part, for such non-living entities.

Headings provided herein are for convenience and are not to be taken as limiting the disclosure in any way.

The enumerated listing of items does not imply that any or all of the items are mutually exclusive, unless expressly specified otherwise.

It is understood that the use of specific component, device and/or parameter names are for example only and not meant to imply any limitations on the invention. The invention may thus be implemented with different nomenclature/terminology utilized to describe the mechanisms/units/structures/components/devices/parameters herein, without limitation. Each term utilized herein is to be given its broadest interpretation given the context in which that term is utilized.

Terminology. The following paragraphs provide definitions and/or context for terms found in this disclosure (including the appended claims):

“Comprising.” This term is open-ended. As used in the appended claims, this term does not foreclose additional structure or steps. Consider a claim that recites: “A memory controller comprising a system cache . . . .” Such a claim does not foreclose the memory controller from including additional components (e.g., a memory channel unit, a switch).

“Configured To.” Various units, circuits, or other components may be described or claimed as “configured to” perform a task or tasks. In such contexts, “configured to” or “operable for” is used to connote structure by indicating that the mechanisms/units/circuits/components include structure (e.g., circuitry and/or mechanisms) that performs the task or tasks during operation. As such, the mechanisms/unit/circuit/component can be said to be configured to (or be operable) for perform(ing) the task even when the specified mechanisms/unit/circuit/component is not currently operational (e.g., is not on). The mechanisms/units/circuits/components used with the “configured to” or “operable for” language include hardware—for example, mechanisms, structures, electronics, circuits, memory storing program instructions executable to implement the operation, etc. Reciting that a mechanism/unit/circuit/component is “configured to” or “operable for” perform(ing) one or more tasks is expressly intended not to invoke 35 U.S.C. .sctn.112, sixth paragraph, for that mechanism/unit/circuit/component. “Configured to” may also include adapting a manufacturing process to fabricate devices or components that are adapted to implement or perform one or more tasks.

“Based On.” As used herein, this term is used to describe one or more factors that affect a determination. This term does not foreclose additional factors that may affect a determination. That is, a determination may be solely based on those factors or based, at least in part, on those factors. Consider the phrase “determine A based on B.” While B may be a factor that affects the determination of A, such a phrase does not foreclose the determination of A from also being based on C. In other instances, A may be determined based solely on B.

The terms “a”, “an” and “the” mean “one or more”, unless expressly specified otherwise.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” and “consisting of” limits the scope of a claim to the specified elements or method steps, plus those that do not materially affect the basis and novel characteristic(s) of the claimed subject matter (see Norian Corp. v Stryker Corp., 363 F.3d 1321, 1331-32, 70 USPQ2d 1508, Fed. Cir. 2004). Moreover, for any claim of the present invention which claims an embodiment “consisting essentially of” or “consisting of” a certain set of elements of any herein described embodiment it shall be understood as obvious by those skilled in the art that the present invention also covers all possible varying scope variants of any described embodiment(s) that are each exclusively (i.e., “consisting essentially of”) functional subsets or functional combination thereof such that each of these plurality of exclusive varying scope variants each consists essentially of any functional subset(s) and/or functional combination(s) of any set of elements of any described embodiment(s) to the exclusion of any others not set forth therein. That is, it is contemplated that it will be obvious to those skilled how to create a multiplicity of alternate embodiments of the present invention that simply consisting essentially of a certain functional combination of elements of any described embodiment(s) to the exclusion of any others not set forth therein, and the invention thus covers all such exclusive embodiments as if they were each described herein.

With respect to the terms “comprising,” “consisting of,” and “consisting essentially of,” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of”, and thus, for the purposes of claim support and construction for “consisting of” format claims, such replacements operate to create yet other alternative embodiments “consisting essentially of” only the elements recited in the original “comprising” embodiment to the exclusion of all other elements.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

As used herein terpene is a naturally occurring hydrocarbon based on combinations of isoprene units. Terpenoids are compounds related to terpenes, which may include some oxygen functionality or some rearrangement. The two terms may be used interchangeably.

In one embodiment, is provided an ester composition prepared using terpenoids and neurotransmitter amino acids. The esters may possess analgesic and anti-inflammatory effects. In one embodiment, is provided pharmaceutical formulations consisting of the ester compositions.

In one embodiment, is provided a composition having a compound of formula I:

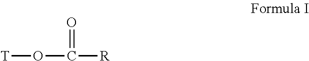

Formula I is an ester of a neurotransmitter amino acid and a terpene, wherein a carboxylic acid group if the neurtransmitter a minor acid is converted to an ester, and wherein T is a derivative of the terpene and R is a derivative of the neurotransmitter amino acid.

In one embodiment, exemplary terpenoids may include, but are not limited to, monoterpenoids, sesquiterpenoiods, diterpenoids, sesterterpenoids, and triterpenoids. Suitable examples of monoterpenes include, but are not limited to, pinene, nerol, citral, camphor, menthol, and limonene. Suitable examples of sesquiterpenoids include, but are not limited to, paradisiol, zingiberol, nerolidol, patchoulol and farnesol. Suitable examples of diterpenoids include, but are not limited to, phytol and retinol. Suitable examples of sesterterpenoids include but are not limited to, geranylfarnesol. Suitable examples of triterpenoids include, but are not limited to, cholesterol, cycloartenol, lanosterol, etc.

In one embodiment, the neurotransmitter amino acids may include, but are not limited to GABA, glycine, and taurine.

It may be appreciated by a person with ordinary skill in the art, in light of and in accordance with the teachings of the present invention, that the administration of the ester composition including compound having Formula I may be performed by any means and methods that may be used to administer the compounds possessing analgesic and anti-inflammatory activity. Suitable methods of administration include, but are not limited to oral, transdermal, topical, rectal, sublingual, and the like. In one embodiment, method of administration may be topical or oral. It may be appreciated by a person with ordinary skill in the art, in light of and in accordance with the teachings of the present invention, that topical and oral methods may have advantages including, but not limited to, direct action and, consequently, reduction of systematic side-effects for topical route; and convenience, ease, and economic advantages for oral administration. It may be appreciated by a person with ordinary skill in the art, in light of and in accordance with the teachings of the present invention, that the following dosage forms may be applied to deliver the ester composition including the formula I, for example, in the form of pills, tablets, capsules, syrups, liquid solutions or suspensions, powders, creams, gels, liniments or balms, lotions or ointment, skin patch, suppositories, etc. . . .

In one embodiment, the ester composition including compound having Formula I may be formulated for oral administration in combination with any non-toxic excipients that are available for the pharmaceutical industry. It may be appreciated by a person with ordinary skill in the art, in light of and in accordance with the teachings of the present invention, that selection and use of relevant excipients as well as providing the required dosage form will depend on the ester used and the recipient. For example, with reference to ester 5-isopropyl-2-methylphenyl 2-aminoacetate hydrochloride, a daily dose (orally) in the range of 1 to 20 mg per kilogram of body weight is preferable when used for pain relief.

In one embodiment, the ester composition including compound having Formula I may be formulated for topical administration in combination with any pharmaceutically acceptable compound. It may be appreciated by a person with ordinary skill in the art, in light of and in accordance with the teachings of the present invention, that the following dosage forms may be applied to deliver the ester composition including the formula I, for example, in the form of ointments, gels, lotions, pastes, solutions, etc. . . . . In an exemplary embodiment, the ointment preparation may include, but not be limited to, lanolin, petrolatum, paraffin, polyethylene glycol, stearic acid, carnauba wax, cetyl alcohol, emulsifying wax, hydrogenated castor oil, etc.

In one embodiment, the amount of ester composition including compound having Formula I in a topical composition may be in a range of from about 0.5 percent to about 20 percent weight by weight based on a total weight of the topical composition. In another embodiment, the amount of ester composition including compound having Formula I in a topical composition may be in a range of from about 1.0 percent to about 15 percent weight by weight based on a total weight of the topical composition. In yet another embodiment, the amount of ester composition including compound having Formula I in a topical composition may be in a range of from about 2.0 percent to about 5 percent weight by weight based on a total weight of the topical composition.

It may be appreciated by a person with ordinary skill in the art, in light of and in accordance with the teachings of the present invention, that use of the ester compositions having compound of formula I of the present invention includes but is not limited to the particular etiology of pain and inflammation caused by insect bites, mechanical injuries, chemical and thermal burn, and the like.

### EXPERIMENTAL SECTION

### Example 1: A Composition for Topical Administration Containing a Compound of Formula 1

This example described the synthesis of (1R,2S,5R)-2-isopropyl-5-menthylcyclohexyl-4-aminobutyrate hydrochloride, a compound having Formula I.

To a stirred solution of 1-menthol (0.5 g, 3.2 mmol) in CH₂Cl₂ (20 mL) at the room temperature Boc-protected GABA (0.662 g, 3.26 mmol) and 4-dimethylaminopyridine (DMAP) (0.097 g, 0.794 mmol) were added. Reaction mass was cooled to 0° C., stirred for 10 min, and N,N′-dicyclohexylcarbodiimide (DCC) was added dropwise (0.727 g, 3.53 mmol). Stirring was continued for 30 min, then the flask was gradually warmed to the room temperature and the stirring continued for additional 10 h. Reaction completion was monitored by TLC. Reaction mixture was filtered, the filtrate was diluted to 100 mL and washed with 1 M aqueous HCl, 10% aqueous NaHCO₃, and water. Deprotection of the N-Boc group was carried out using HCl/CH₃COOH. The crude product was recrystallized from an appropriate solvent affording the desired ester. Yield: 96%. Previously we have patented the compound (patent of Ukraine No 112035) with the following structural formula:

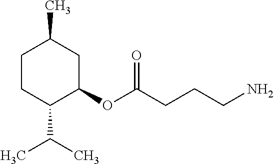 [SMILES: CC(C)[C@@H]1CC[C@@H](C)C[C@H]1OC(=O)CCCN]

named (1R,2S,5R)-2-isopropyl-5-methylcyclohexyl 4-aminobutyrate which possess anticonvulsant and sedative activity.

The abovementioned structural formula of patented compound is in complete agreement with the general structural formula of compounds claimed in the present patent:

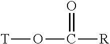

### Example 2: A Composition for Topical Administration Containing a Compound of Formula 1

The composition is prepared by combining the following ingredients:

5-isopropyl-2-methylphenyl 4-aminobutyrate hydrochloride, 0.18 g, polyethylene glycol (PEG 1500, 4.0 g); polyethylene oxide (PEO 400, 2.0 g); and 1,2-propylene glycol (3.0 g)

In a 50 milliliters cylindrical/round bottom flask was added polyethylene glycol, polyethylene oxide, and 1,2-propylene glycol. A resultant mixture was heated to a temperature of about 90 to about 100 degrees Celsius in period of about 10 min minutes to provide a melted mixture. The ester 5-isopropyl-2-methylphenyl 4-aminobutyrate hydrochloride was added to this melted mixture (at the melting temperature of PEG 1500). The resultant mixture was stirred thoroughly, and cooled to a temperature of about 0 degrees Celsius to provide an ointment. The resultant ointment was found to contain about 2 percent by weight of 5-isopropyl-2-methylphenyl 4-aminobutyrate hydrochloride. The ointment may be stored under refrigeration around +3 degrees Celsius to prevent any appreciable loss of potency.

### Example 3: Analgesic Effect Effectiveness of the Formulations Prepared Using the Process Outlined in Example 1 was Evaluated for Topical Application of the Formulation

Example 3 illustrates the analgesic activity of the esters evaluated using models of providing thermal stimuli (hot plate) and chemical stimuli (formalin, capsaicin and allyl isothiocyanate) to the subjects i.e., mice.

TEST 1: Hot plate test: The hot plate test (HPT) is often used for investigation of a noxious stimulus (heat) in models of acute pain. Classically, the latency of the animal's response to the stimulus is recorded as the dependent variable.

In this test groups of 6 male mice were used in order to evaluate the analgesic activity of the compounds listed in Table 1. The analgesic activity of compounds was comparable with a reference drug benzocaine (BZC), which is a local anesthetic commonly used as a topical pain reliever. All compounds, including BZC, were applied topically in an ointment (2 percent weight by weight based on the total weight of formulation) prepared according to the procedure described in Example 1. The ointments were applied both to the palmar and plantar surfaces of the paws of the mice by gently rubbing 5 min before analgesic activity evaluation. A corresponding amount of the ointment base were similarly applied for mice used as control animals. The mice were placed on a hot plate maintained at 55 degrees Celsius, one at a time. In this experiment, latency to respond to the heat stimulus was determined by the amount of time (in seconds) it took for a mouse to lick one of its paws. Cut-off time was fixed at 60 seconds to minimize the tissue damage that occurs during prolonged contact with heated surface. All values are expressed as the mean±standard errors of the mean (SEM) in all the tables. The results are summarized in TABLE 1 below:

TABLE 1

| | Reaction time |
|---|---|
| Compound | (in sec) |
| Control | 10.3 ± 0.6 |
| Benzocaine | 18.3 ± 0.9 |
| COMPOUND 1: (1R,2S,5R)-2-isopropyl-5- | 30.2 ± 2.6 |
| methylcyclohexyl-4-aminobutyrate hydrochloride | |
| COMPOUND 2: 2-isopropyl-5-methylphenyl 4- | 21.3 ± 0.9 |
| aminobutyrate hydrochloride | |
| COMPOUND 3: 5-isopropyl-2-methylphenyl 4- | 19.3 ± 3.8 |
| aminobutyrate hydrochloride | |
| COMPOUND 4: 2-methoxylphenyl 4-aminobutyrate | 16.5 ± 3.7 |
| hydrochloride | |

As seen in Table 1, the control animals that received only an ointment base with no analgesic started licking their paws for pain with about 10.3 seconds of being placed on the hotplate. The animals that received BZC as the analgesic started licking their paws for pain with about 18.3 seconds of being placed on the hotplate. The animals that received (1R,2S,5R)-2-isopropyl-5-methyl cyclohexyl-4-aminobutyrate hydrochloride as the analgesic started licking their paws for pain with about 30.2 seconds of being placed on the hotplate. The animals that received 2-isopropyl-5-methylphenyl 4-aminobutyrate hydrochloride as the analgesic started licking their paws for pain with about 21.3 seconds of being placed on the hotplate. The animals that received 5-isopropyl-2-methylphenyl 4-aminobutyrate hydrochloride as the analgesic started licking their paws for pain with about 19.3 seconds of being placed on the hotplate. The animals that received 2-methoxylphenyl 4-aminobutyrate hydrochloride as the analgesic started licking their paws for pain with about 16.5 seconds of being placed on the hotplate. The animals that received Compound 1 to Compound 4 prepared in accordance with the instant invention showed relatively better or comparable pain resistance to BZC i.e., compound 1 about 30.2 seconds, compound 2 about 21.3 seconds, compound 3 about 19.3 seconds, and compound 4 about 16.5 seconds.

TEST 2: Capsaicin test: Capsaicin (8-methyl-N-vanillyl-6-nonenamide), the pungent substance obtained from hot red pepper, is used to evaluate analgesic activity on mice, for compounds synthesized herein.

All compounds, including BZC, were applied topically as an ointment (2 percent weight by weight based on the total weight of formulation) prepared according to the procedure described in Example 2. The ointments were applied to the affected paws' surface by gently rubbing 5 min before analgesic activity evaluation. A corresponding amount of the ointment base were similarly applied for mice used as control animals. To evaluate analgesic activity 20 microliter of capsaicin solution in 1,2-propylene glycol (6 micrograms/paw) was injected subcutaneously under the skin of the dorsal surface of the hindpaw. Animals were observed individually for 5 minutes after capsaicin administration. The amount of time that mice spent licking the injected paw was recorded and considered as an index of pain. The results are summarized in TABLE 2 below:

TABLE 2

| | Licking time |
|---|---|
| Compound | (in sec) |
| Control | 45.7 ± 1.8 |
| Benzocaine | 28.7 ± 6.6 |
| (1R,2S,5R)-2-isopropyl-5-methylcyclohexyl-4- | 4.3 ± 0.9 |
| aminobutyrate hydrochloride | |
| 2-isopropyl-5-methylphenyl 4-aminobutyrate hydrochloride | 19.7 ± 2.6 |
| 5-isopropyl-2-methylphenyl 4-aminobutyrate hydrochloride | 12.0 ± 1.2 |
| 2-methoxylphenyl 4-aminobutyrate hydrochloride | 24.3 ± 3.3 |

TEST 3: Formalin test: The formalin test was used to determine the potential analgesic effects of compounds for states of persistent pain in which tissue damage occurs. The nociceptive response produced by formalin is biphasic: an early response (0 to 5 min after injection) and a late response (20 to 30 minutes after injection). These two phases can be distinguished pharmacologically: phase 1 is supposed to represent a direct chemical stimulation of nociceptors while phase 2 is thought to involve inflammation. Aiming at assessment precisely response caused by a burst of activity from pain fibers we measured the licking time as an indicator of nociception only during the first (early) phase. All compounds, including BZC, were applied topically in an ointment (2 percent weight by weight based on the total weight of formulation) prepared according to the procedure described in Example 2. The ointments were applied to the affected paws' surface by gently rubbing 5 minutes before analgesic activity evaluation. A corresponding amount of the ointment base were similarly applied for mice used as control animals. After that, 20 microliter of a 2 percent formalin solution in 0.9 percent NaCl (w/w) was injected subcutaneously into the dorsal surface of the hindpaw. The animal then was placed in an individual plexiglass cage. The time spent by the animal in licking the injected paw was measured from 0 to 5 min (early phase) after formalin administration and was considered as an indicator of pain response. The results are summarized in TABLE 3 below:

TABLE 3

| | Licking time |
|---|---|
| Compound | (in sec) |
| Control | 103.5 ± 8.5 |
| Benzocaine | 36.3 ± 2.4 |
| (1R,2S,5R)-2-isopropyl-5-methylcyclohexyl-4- | 30.3 ± 7.4 |
| aminobutyrate hydrochloride | |
| 2-isopropyl-5-methylphenyl 4-aminobutyrate | 52.2 ± 6.6 |
| hydrochloride | |
| 5-isopropyl-2-methylphenyl 4-aminobutyrate | 31.1 ± 8.3 |
| hydrochloride | |
| 2-methoxylphenyl 4-aminobutyrate hydrochloride | 47.6 ± 9.8 |

TEST 4: Allyl isothiocyanate test: The analgesic activity was evaluated by a model of chemical stimulus with the use of TRPA1 agonist—allyl isothiocyanate (AITC).

20 Microliters of 0.5 percent (w/w in 1,2-propylene glycol) AITC solution was injected subcutaneously under the skin of the dorsal surface of the right hindpaw. All compounds, including BZC, were applied topically in an ointment (2 percent weight by weight based on the total weight of formulation) prepared according to the procedure described in Example 2. The ointments were applied to the affected paws' surface by gently rubbing 5 min before analgesic activity evaluation. A corresponding amount of the ointment base were similarly applied for mice used as control animals. AITC dose was selected based on our preliminary trials and from literature citations. Animals were observed individually for 10 minutes after AITC administration. The amount of time that mice spent licking the injected paw (reaction time in seconds) was recorded and considered as an index of pain. The results are summarized in TABLE 4:

TABLE 4

| | Licking time |
|---|---|
| Compound | (in sec) |
| Control | 71.3 ± 1.8 |
| Benzocaine | 48.0 ± 2.0 |
| (1R,2S,5R)-2-isopropyl-5-methylcyclohexyl-4- | 2.7 ± 0.3 |
| aminobutyrate hydrochloride | |
| 2-isopropyl-5-methylphenyl 4-aminobutyrate | 20.3 ± 5.8 |
| hydrochloride | |
| 5-isopropyl-2-methylphenyl 4-aminobutyrate | 22.8 ± 4.3 |
| hydrochloride | |
| 2-methoxylphenyl 4-aminobutyrate hydrochloride | 25.3 ± 1.5 |

### Example 3: Analgesic Effect Effectiveness of the Formulation (Analgesic Composition for Oral Administration) Prepared was Evaluated for Oral Administration of the Formulation

The analgesic composition for oral administration was prepared using Tween® 80/water emulsion (1:10 weight/weight), and 5-isopropyl-2-methylphenyl 2-aminoacetate hydrochloride. 5-isopropyl-2-methylphenyl 2-aminoacetate hydrochloride was dissolved in Tween® 80/water emulsion (1:10 w/w) and administered to mice orally in the range of doses 10 to 200 milligrams per kilogram of body weight of a mouse. The analgesic action was then determined in the model of chemically-induced pain with the use of capsaicin as described in Example 2 Test 2. The results are summarized in TABLE 5 below:

TABLE 5

| Dose, mg/kg | Licking time, secs |
|---|---|
| 10 | 28.8 ± 2.3 |
| 25 | 21.5 ± 0.5 |
| 50 | 7.3 ± 0.6 |
| 100 | 9.8 ± 0.4 |
| 200 | 7.7 ± 2.1 |
| Control | 45.7 ± 1.8 |

### Example 5: Anti-Inflammatory Effect Effectiveness of the Formulation Prepared was Evaluated for Inflammation Reducing

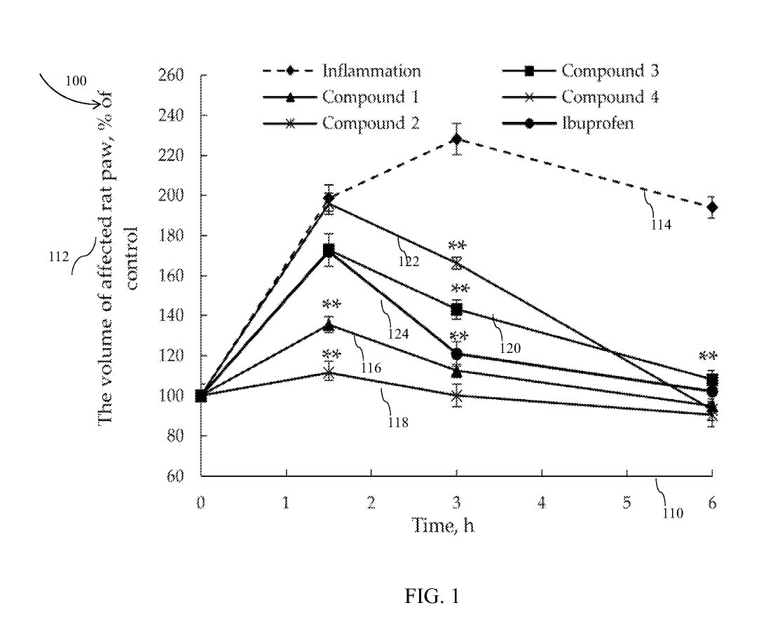

Referring to FIG. 1 is illustrated a graph 100 providing the comparison of anti-inflammatory effect between the compounds 1 to 4 prepared in accordance with the present invention and a reference drug ibuprofen. Accordingly, FIG. 1 illustrates an exemplary graph of the anti-inflammatory activity of esters designated by ester compounds 1-4 on the model of AITC-induced rat paw edema. The appropriate numbers are assigned to the following esters: (1R,2S,5R)-2-isopropyl-5-methylcyclohexyl-4-aminobutyrate hydrochloride—compound 1; 2-isopropyl-5-methylphenyl 4-aminobutyrate hydrochloride—compound 2; 5-isopropyl-2-methylphenyl 4-aminobutyrate hydrochloride—compound 3; 2-methoxylphenyl 4-aminobutyrate hydrochloride—compound 4. This FIGURE is time dependence of the volume of affected rat paw after topical delivery (2 percent weight by weight ointment) of esters 1-4 and ibuprofen (reference drug). ** p<0.01 compared with vehicle-treated group. One-way analysis of variance (ANOVA) followed by Tukey's post hoc comparison.

As shown in the graph, time in hours is plotted on the X-Axis 110, and the volume of the effected rat paw treated with the anti-inflammatory compounds 1 to 4 and ibuprofen as a percentage of the control is plotted on the Y-Axis 112. The anti-inflammatory effect was determined using an AITC-induced rat paw edema assay. All compounds, including ibuprofen, were applied topically in an ointment (2 percent weight by weight based on the total weight of formulation) prepared according to the procedure described in Example 2. The ointment was applied 30 minutes prior to edema inducing by subplantar injection of 30 microliters of an AITC solution (100 microgram per paw) in propylene glycol. Edema was measured at 0 hours, 1.5, 3, 6 and 24 hours after AITC injection by using a plethysmometer. The ointments were additionally applied by gently rubbing on the affected paw at each time point.

It may be appreciated by a person with ordinary skill in the art, in light of and in accordance with the teachings of the present invention, that the activation of TRPA1 channels by AITC leads not only to the development of hyperalgesia but may also results in inflammatory edema. To examine the anti-inflammatory activity of compounds 1 to 4, i.e., GABA esters 1-4, AITC was injected into the dorsal surface of the rat paw at a dose selected by studying of the dose-response relationship—100 microgram per paw. Ibuprofen, which according to the literature data inhibits inflammatory edema evoked by AITC, was selected as a reference drug. As shown in FIG. 1, AITC-induced edema, indicated by line 114 in the graph, was maximum at 3 hours and decreased to the base level (105 percent ±3.9 percent, data not shown in graph after 6 hours) to 24 hours after the injection. As seen, after transdermal delivery (2 percent weight/weight ointment) of compounds 1, indicated by line 116, compound 2 indicated by line 118, compound 3 indicated by line 120, compound 4 indicated by line 122 and ibuprofen indicated by line 124, the inflammation maximum was observed after about 1.5 hours following AITC injection. Our data indicate that the volume of affected rat paw used as a measure of edema (in percentage) reached the basal level (100 percent) at about 6 hours after AITC administration.

Topical application of all studied esters significantly reduced the inflammation level compared with vehicle-treated group of rats (inflammation curve). The highest anti-inflammatory action was demonstrated by compounds 1 116 and 2 118 which after topical application significantly decreased the level of AITC-induced edema to 136 percent and 112 percent, respectively; while the treatment with ibuprofen ointment reduced the volume of affected paw to 172 percent. Thus, esters 1 and 2 were found to exceed reference drug ibuprofen by their ability to decrease the inflammatory state induced by intraplantar injection of AITC, a TRPA1 activator while esters 3 and 4 were found to have relatively comparable effects with ibuprofen.

Those skilled in the art will readily recognize, in light of and in accordance with the teachings of the present invention, that any of the foregoing steps may be suitably replaced, reordered, removed and additional steps may be inserted depending upon the needs of the particular application. Moreover, the prescribed method steps of the foregoing embodiments may be implemented using any physical and/or hardware system that those skilled in the art will readily know is suitable in light of the foregoing teachings. For any method steps described in the present application that can be carried out on a computing machine, a typical computer system can, when appropriately configured or designed, serve as a computer system in which those aspects of the invention may be embodied. Thus, the present invention is not limited to any particular tangible means of implementation.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC §112 (1), all claims must be supported by sufficient disclosure in the present patent specification, and any material known to those skilled in the art need not be explicitly disclosed. However, 35 USC §112 (6) requires that structures corresponding to functional limitations interpreted under 35 USC §112 (6) must be explicitly disclosed in the patent specification. Moreover, the USPTO's Examination policy of initially treating and searching prior art under the broadest interpretation of a “mean for” claim limitation implies that the broadest initial search on 112(6) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC §112 (6) when such corresponding structures are not explicitly disclosed in the foregoing patent specification. Therefore, for any invention element(s)/structure(s) corresponding to functional claim limitation(s), in the below claims interpreted under 35 USC §112 (6), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed. Applicant(s) request(s) that fact finders during any claims construction proceedings and/or examination of patent allowability properly identify and incorporate only the portions of each of these documents discovered during the broadest interpretation search of 35 USC §112 (6) limitation, which exist in at least one of the patent and/or non-patent documents found during the course of normal USPTO searching and or supplied to the USPTO during prosecution. Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3^{rd} parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC §112 (6), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC §112 (6). Applicant(s) note that all the identified documents above which are incorporated by reference to satisfy 35 USC §112 (6) necessarily have a filing and/or publication date prior to that of the instant application, and thus are valid prior documents to incorporated by reference in the instant application.

Having fully described at least one embodiment of the present invention, other equivalent or alternative ester compositions having a compound of formula I and methods of preparing the same according to the present invention will be apparent to those skilled in the art. Various aspects of the invention have been described above by way of illustration, and the specific embodiments disclosed are not intended to limit the invention to the particular forms disclosed. The particular implementation of the ester compositions having a compound of formula I and methods of preparing the same may vary depending upon the particular context or application. By way of example, and not limitation, the ester compositions having a compound of formula I and methods of preparing the same described in the foregoing were principally directed to analgesic and anti-inflammatory implementations; however, similar techniques may instead be applied to which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. That is, the Abstract is provided merely to introduce certain concepts and not to identify any key or essential features of the claimed subject matter. It is submitted with the understanding that it will not be used to limit or interpret the scope or meaning of the claims.

The following claims are hereby incorporated into the detailed description, with each claim standing on its own as a separate embodiment.

1. A composition comprising:
a compound having a formula I: 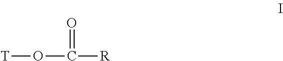
wherein T is derivative of a terpene and R is a derivative of a neurotransmitter acids, wherein the neurotransmitter acids are selected form GABA, glycine, and taurine.

2. The composition of claim 1, wherein T is derived from 1-menthol.

3. A formulation comprising:
at least one compound having a formula I: 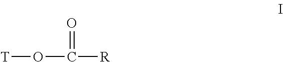
wherein T is derivative of a terpene and R is a derivative of a neurotransmitter acids, wherein the neurotransmitter acids are selected form GABA, glycine, and taurine;
at least one free monoterpenoid; and
at least one pharmaceutically acceptable base;
wherein the formulation is an analgesic and anti-inflammatory pharmaceutical composition for topical application.

4. A formulation comprising:
at least one compound having a formula I: 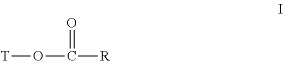
wherein T is derivative of a terpene and R is a derivative of a neurotransmitter acids, wherein the neurotransmitter acids are selected form GABA, glycine, and taurine;
at least one free monoterpenoid;
and
at least one pharmaceutically acceptable excipient;
wherein the formulation is an analgesic and anti-inflammatory pharmaceutical composition for oral use.

5. A formulation comprising:
at least one compound having a formula I: 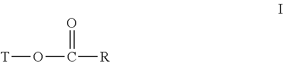
wherein T is derivative of a terpene and R is a derivative of a neurotransmitter acids, wherein the neurotransmitter acids are selected form GABA, glycine, and taurine; wherein the compound of formula I is present in an amount in a range of from about 0.5 percent to about 20 percent by weight of the total formulation; and
at least one pharmaceutically acceptable excipient;
wherein the formulation is an analgesic and anti-inflammatory pharmaceutical composition for topical use.
